(19) United States
(12) Patent Application Publication
(10) Pub. No.: US 2025/0010072 A1
(43) Pub. Date: Jan. 9, 2025

(54) SYSTEM AND METHOD FOR A MICRO STIMULATION DEVICE FOR OCULAR TREATMENT

(71) Applicant: Andy Rosenfarb, Westfield, NJ (US)

(72) Inventor: Andy Rosenfarb, Westfield, NJ (US)

(21) Appl. No.: 18/764,954

(22) Filed: Jul. 5, 2024

Related U.S. Application Data
(60) Provisional application No. 63/525,057, filed on Jul. 5, 2023.

(51) Int. Cl. A61N 1/36; A61N 1/04

(52) U.S. Cl. CPC A61N 1/36034; A61N 1/0456; A61N 1/0472; A61N 1/36046

(57) ABSTRACT

A portable, handheld device and electrodes deliver precise electrical stimulation to targeted areas around the skin of the face and eyelids to treat ocular conditions. The micro stimulation device generates microcurrent using a current generator. One or more electrodes are electrically coupled to the micro stimulation device and contact and conduct the microcurrent to the facial and/or ocular skin of a patient. The current generator is configured to generate the microcurrent at a selected one of a plurality of power levels, wherein the plurality of power levels includes: 250 microamps (μA), 500 μA, 750 μA, 1000 μA, 1250 μA, and 1500 μA. A first user input is configured for a user to select one of the plurality of power levels.

### CROSS REFERENCE TO RELATED APPLICATION

This application claims priority under 35 U.S.C. § 119 to U.S. Provisional Application No. 63/525,057 entitled, “SYSTEM AND METHOD FOR A MICRO STIMULATION DEVICE FOR OCULAR TREATMENT,” filed Jul. 5, 2023, and hereby expressly incorporated by reference herein.

### COPYRIGHT NOTICE

A portion of the disclosure of this patent document contains material which is or may be subject to copyright protection. The copyright owner has no objection to the facsimile reproduction by anyone of the patent document or the patent disclosure, as it appears in the Patent and Trademark Office patent file or records, but otherwise reserves all copyrights whatsoever.

### FIELD

The present application relates generally to devices and methods for micro stimulation therapy, and more particularly to therapeutic, at home devices and methods that provide microcurrent stimulation to cutaneous tissue in or around an eye region for the treatment of macular degeneration and/or other ocular diseases.

### BACKGROUND

Micro stimulation is a technique to apply electrical stimulation to nerve fibers using cutaneous electrodes positioned on the skin of a patient. One method of providing electrical stimulation therapy is to deliver current to tissue on or near the area of the body to be treated. For example, microcurrents in the range of 100 microamps to 1,000 microamps (peak) have been applied to tissue. These electrotherapeutic devices are very limited in scope with respect to output (i.e., only output a current having a single amplitude) and capabilities. In addition, currently, micro stimulation devices are only found in professional health care settings and treatments are only conducted by health care providers.

Electrostimulation therapy to prevent or reverse diseases of the eye is of great interest. As life expectancy expands, more and more of the population is at risk for age related macular degeneration (AMD). Meanwhile, smaller populations of young patients suffer from a variety of maladies that affect the retina and other structures of the eye. A wide variety of other vision disorders exist which can lead to partial or total blindness. There is a continuing demand for new and alternative systems and methods to treat such disorders.

Thus, there is a need for an improved micro stimulation device and method that may be used in a convenient at home setting to treat eye conditions.

### SUMMARY OF THE INVENTION

In one or more aspects herein, a micro stimulation device includes a current generator configured to generate a current; and at least a first electrode electrically coupled to the micro stimulation device, wherein the at least first electrode is configured to contact and conduct the microcurrent to facial and/or ocular skin of a patient.

In one or more aspects herein, the current generator is configured to generate the microcurrent at a selected one of a plurality of power levels, wherein the plurality of power levels includes: 250 microamps (μA), 500 μA, 750 μA, 1000 μA, 1250 μA, and 1500 μA.

In one or more aspects herein, the current generator is configured to generate the microcurrent at a selected one of a plurality of power levels, wherein the plurality of power levels includes: 250 microamps (μA), 500 μA, 750 μA, and 1000 μA.

In one or more aspects herein, the micro stimulation device includes a first user input control configured for a user to select one of the plurality of power levels.

In one or more aspects herein, the micro stimulation device includes a control unit including a processing circuit and a memory device. The memory device stores instructions that, when executed by the at least one processor, causes the micro stimulation device to determine the selected one of the plurality of power levels from the first user input control; and control the current generator to generate the microcurrent at the selected one of the plurality of power levels.

In one or more aspects herein, the micro stimulation device includes a display, and a graphical user interface generated on the display that indicates the selected one of the plurality of power levels.

In one or more aspects herein, the micro stimulation device includes a second user input control configured for a user to select one of a plurality of treatment periods, wherein the plurality of treatment periods includes: 10 minutes, 20 minutes, and 30 minutes.

In one or more aspects herein, the control unit is further configured to cause the micro stimulation device to determine the selected one of the plurality of treatment periods from the second user input control and control the current generator to generate the microcurrent for a duration of the selected one of the plurality of time settings.

In one or more aspects herein, the control unit is further configured to cause the micro stimulation device to generate a graphical user interface on the display that indicates the selected one of the plurality of treatment periods.

In one or more aspects herein, the micro stimulation device includes a rechargeable battery configured to power the micro stimulation device, and a power charge port that is configured to connect to a power source and charge the rechargeable battery.

In one or more aspects herein, the micro stimulation device includes a microcurrent output port coupled to the current generator, wherein the microcurrent output port is coupled to the at least first electrode using a first lead wire.

In one or more aspects herein, the micro stimulation device includes at least a second electrode and a second lead wire coupled to the microcurrent output port and the second electrode.

In one or more aspects herein, the first electrode and the second electrode each include an electrode cup; and an electrode pad fitted within the electrode cup, wherein the electrode pad is removeable and replaceable from the electrode cup.

In one or more aspects herein, the first electrode is positioned on and stimulates a right temple of a user with the microcurrent, and the second electrode is positioned on and stimulates a left temple of the user with the microcurrent.

In one or more aspects herein, the first electrode is positioned on and stimulates a right eye area of a user with the microcurrent, and the second electrode is positioned on and stimulates a left eye area of the user with the microcurrent.

In one or more aspects herein, the first electrode is positioned on and stimulates a right forehead area of a user with the microcurrent, and the second electrode is positioned on and stimulates a left forehead area of the user with the microcurrent.

In one or more aspects herein, the display includes a graphical user interface that indicates a plurality of power levels, wherein the plurality of power levels includes: 250 micro amps (μA), 500 μA, 750 μA, 1000 μA, 1250 μA, and 1500 μA, and the first user input control is configured for a user to select one of the plurality of power levels on the display.

In one or more aspects herein, the display includes a graphical user interface that indicates a plurality of treatment periods, wherein the plurality of treatment periods includes: 10 minutes, 20 minutes, and 30 minutes, and the second user input control configured for a user to select one of the plurality of treatment periods on the display.

In one or more aspects herein, the micro stimulation device is sized for portability and to be handheld.

In one or more aspects herein, the micro stimulation device has a thickness of 12 mm to 16 mm, a height of 100 mm to 150 mm, and a width of 75 mm to 100 mm.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments that are presently preferred. It should be understood, however, that the claims are not limited to the precise arrangements and instrumentalities shown.

FIGS. 1A-B illustrate a perspective view of an exemplary embodiment of the portable micro stimulation device for treatment of eye conditions.

FIG. 2A-C illustrate a perspective view of another exemplary embodiment of the portable micro stimulation device for treatment of eye conditions.

FIG. 3 illustrates a perspective view of an exemplary embodiment of the electrodes of the micro stimulation device.

FIGS. 4A-C illustrate perspective views of exemplary embodiments of various recommended placements of the electrodes of the micro stimulation device on a user.

FIGS. 5A-B illustrate graphs of an embodiment of the microcurrent generated by the micro stimulation device.

FIG. 6 illustrates a schematic block diagram of an exemplary embodiment of the micro stimulation device.

FIG. 7A illustrates a flow chart of an exemplary embodiment of a method for initiation of operation of the micro stimulation device by a user.

FIG. 7B illustrates a flow chart of an exemplary embodiment of a method of operation of the micro stimulation device.

### DETAILED DESCRIPTION

The word “exemplary” or “embodiment” is used herein to mean “serving as an example, instance, or illustration.” Any implementation or aspect described herein as “exemplary” or as an “embodiment” is not necessarily to be construed as preferred or advantageous over other aspects of the disclosure. Likewise, the term “aspects” does not require that all aspects of the disclosure include the discussed feature, advantage, or mode of operation.

Embodiments will now be described in detail with reference to the accompanying drawings. Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “an appliance” may include a plurality of appliances. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs, and materials are now described. Where there are discrepancies in terms and definitions used in references that are incorporated by reference, the terms used in this application shall have the definitions given herein.

In the following description, numerous specific details are set forth in order to provide a thorough understanding of the aspects described herein. It will be apparent, however, to one skilled in the art, that these and other aspects may be practiced without some or all of these specific details. In addition, well known steps in a method of a process may be omitted from flow diagrams presented herein in order not to obscure the aspects of the disclosure. Similarly, well known components in a device may be omitted from figures and descriptions thereof presented herein in order not to obscure the aspects of the disclosure.

Micro stimulation is a promising treatment option for various ocular conditions. This innovative approach involves the use of a portable, handheld device and electrodes to deliver precise electrical stimulation to targeted areas around the skin of the face and eyelids. The development of a micro stimulation device for at-home ocular treatment represents a significant advancement in the field of ophthalmology. This innovative technology has the potential to revolutionize the treatment of various ocular conditions, offering more convenient and accessible treatment to patients. The improved device described herein is designed to deliver targeted electrical stimulation to cutaneous tissues, with the goal of modulating neural activity. By affecting the neural circuitry of the eye, the micro stimulation device is able to treat a wide range of ocular conditions, including retinal degenerative diseases, optic nerve disorders, and other vision-related ailments. In an embodiment, the electrical stimulation can be tailored in terms of at least intensity and duration, allowing for fine control over the treatment.

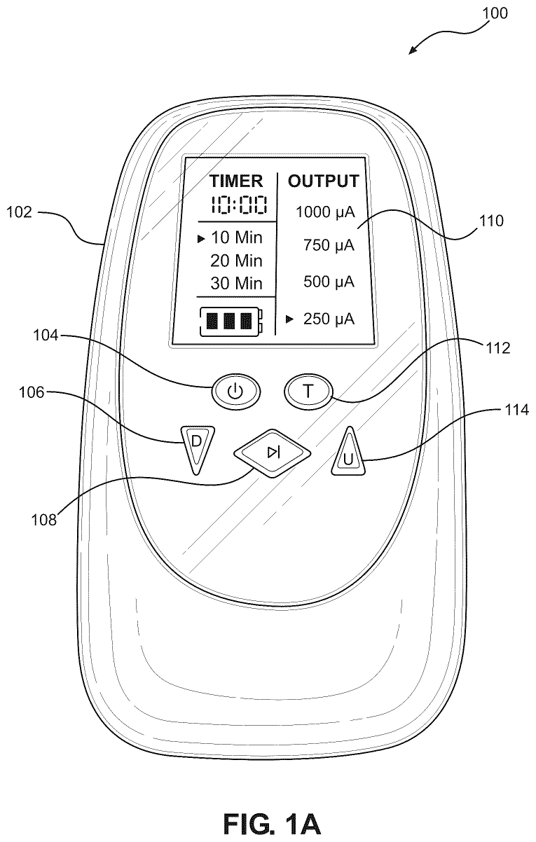

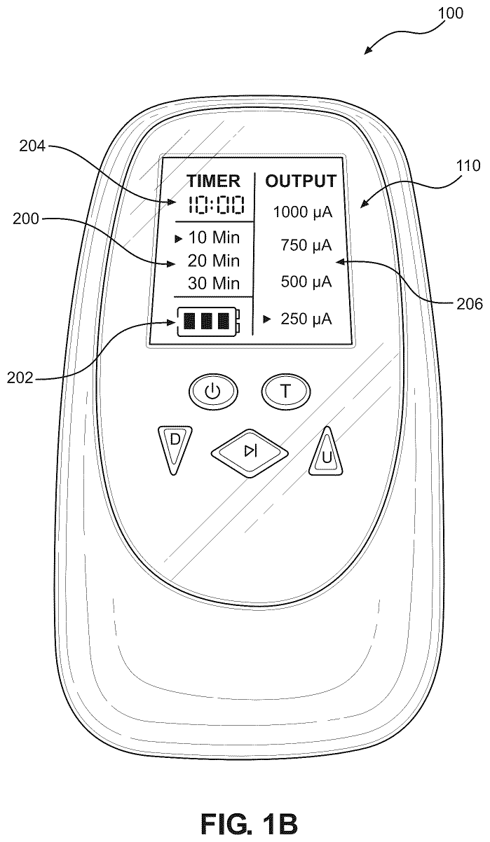

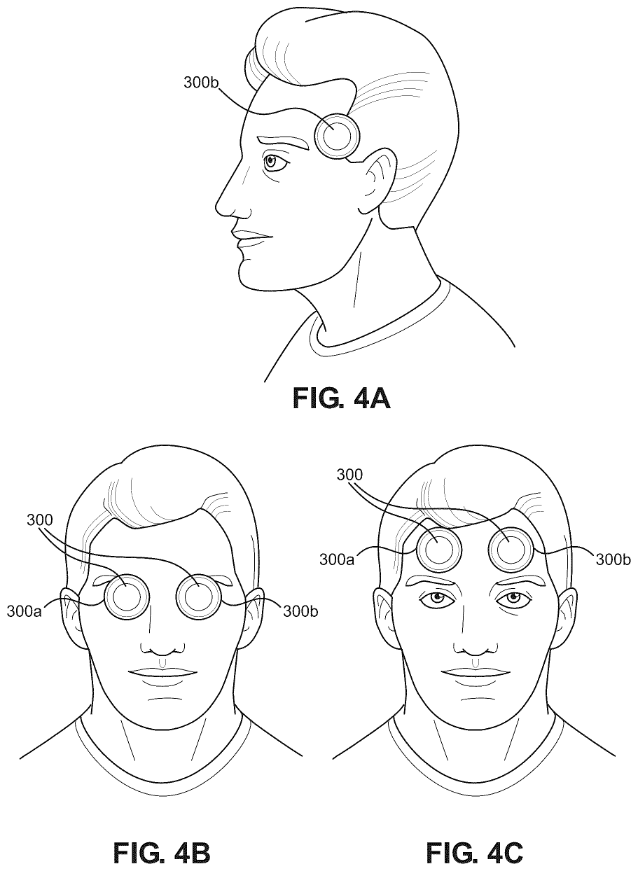

FIGS. 1A and 1B illustrate perspective views of an exemplary embodiment of a portable, at home micro stimulation device 100 for treatment of eye conditions. The micro stimulation device 100 is small, lightweight and portable and thus may be used in home settings by a patient—without the need to visit a clinic or a health care professional. In an embodiment, the micro stimulation device 100 helps in vision recovery. For example, the electrodes 300 positioned over the eyes, forehead, or temples to aid in the recovery of vision related diseases by stimulating the cells to reactivate, grow, and detoxify increasing their viability (as depicted in FIGS. 4A, 4B, and 4C). In another embodiment, the micro stimulation device 100 helps in prevention of ocular degeneration. For those with degenerative eye disease, the micro stimulation device 100 may help prevent further damage of the ocular systems.

The micro stimulation device 100 delivers a low amplitude current to the skin of a patient via electrodes that are positioned on the forehead, temples, or eye area. The low amplitude current may range from 100 microamps (μA) to 1500 μA. Since the device 100 only outputs a low amplitude current, it may be used safely on the skin without burning or other injury to the patient.

In one example, the low amplitude current is a direct current (DC). In another example, the low amplitude current is an alternating current (AC). In yet another example, the low amplitude current may be adjusted between AC and DC or between different AC frequencies, either manually or automatically.

Referring back to FIGS. 1A and 1B, the micro stimulation device 100 includes a user interface that functions to power on/off, start, pause, and increase or decrease the amplitude of the current and the duration of the treatment. In this embodiment, the user interface includes a plurality of buttons on a front panel 102 of the micro stimulation device 100. For example, a power button 104 turns the power of the micro stimulation device 100 on and off. In one embodiment, for safety, the power button 104 must be pressed for 3 seconds to power on the micro stimulation device 100 or power off the micro stimulation device 100.

A time setting button 112 on the front of the micro stimulation device 100 selects one of a plurality of treatment periods. In one example, the treatment periods include 10 minute, 20 minute, and 30 minute periods, though alternate and/or additional treatments periods may be included and selected. The shortest time period of 10 minutes is the default when no other treatment period is selected. The time setting button 112 is pressed/activated by the user to select a different treatment period.

A start/pause button 108 on the front panel 102 of the micro stimulation device 100 controls the activation and deactivation of the micro stimulation current. A user may pause the micro stimulation and restart the micro stimulation during a treatment period using the start/pause button 108.

In one example, an increase button 114 and decrease button 106 select a power level of the micro stimulation device 100. For example, the micro stimulation device 100 may generate a plurality of power levels: 250 μA, 500 μA, 750 μA, 1000 μA, 1250 μA, and 1500 μA. The lowest power setting of 250 μA is the default when no power level is selected by the user. The increase button 114 on the front panel 102 of the micro stimulation device 100 may be pressed/activated to increase the power level, and the decrease button 106 on the front panel 102 of the micro stimulation device 100 may be pressed/activated to decrease the power level. For example, the power level of the current is increased by one level each time the increase button 114 is pressed/activated, and the power level of the current is decreased by one level each time the decrease button 106 is pressed/activated. Though the user controls are shown as various push buttons, other user controls may be implemented, such as a touch screen, turnable knobs, keyboard, touch pad, mouse, or other user interfaces.

The micro stimulation device 100 includes various safety features. For example, the micro stimulation device 100 defaults to the lowest time setting for the treatment period (e.g., 10 mins) and power level (e.g., 250 μA). In addition, when the selected treatment period expires, the micro stimulation device 100 will automatically halt generation of the output current and may also turn off the device 100. The micro stimulation device 100 also automatically halts generation of the output current after a predetermined time period when the start/pause button 108 is pressed to initiate treatment, but the electrodes are not positioned on the patient (e.g., without a detected load). For example, the micro stimulation device 100 automatically powers off after 2 minutes when a completed circuit is not made with the electrodes.

FIG. 1B illustrates a perspective view of an exemplary embodiment of a display screen 110 of the micro stimulation device 100. The display screen 110 may include a liquid crystal display (LCD), In-Plane Switching Liquid Crystal Display (IPS-LCD), Organic Light-Emitting Diode (OLED), Active-Matrix Organic Light-Emitting Diode (AMOLED) or another type of display technology.

The display screen 110 of the micro stimulation device 100 indicates the selected power settings. For example, an arrow is displayed next to 250 μA to indicate the selected power level 206. In addition, the display screen 110 indicates a selected treatment period 200. For example, another arrow is displayed to indicate the selected treatment period 200 is 10 minutes.

The display screen 110 also includes a timer 204 set to the selected treatment period 200. Upon start of the treatment period, the timer 204 begins to count down to indicate the remaining treatment duration. The micro stimulation device 100 may also display a battery charge level indicator 202 of a rechargeable battery. A user may thus know when the battery charge is low and plug the micro stimulation device 100 into a power source.

In another embodiment, the display screen 110 may include a touchscreen and one or more graphical user interfaces (GUIs) may be generated to select the settings of the micro stimulation device 100. For example, one or more GUIs may be generated by the micro stimulation device 100, e.g., using Python or other GUI programming, that allow selection of one of the plurality of power levels. The one or more GUIs may also provide selection of a treatment period 200, such as 10, 20, or 30 minutes.

The micro stimulation device 100 is sized for portability and to be handheld. For example, the micro stimulation device 100 has a thickness of 12 mm to 16 mm, a height of 100 mm to 150 mm, and a width of 75 mm to 100 mm. The micro stimulation device 100 may also be lightweight, such as weighing between 0.5 lbs to 3 lbs. In addition, the user interface is designed for ease of use by a non-professional. The settings, such as the low microcurrent power levels, are designed to prevent injury to a user. These advantages allow the micro stimulation device 100 to be used safely and easily at home by a non-medical professional while still maintaining effective treatment of ocular conditions.

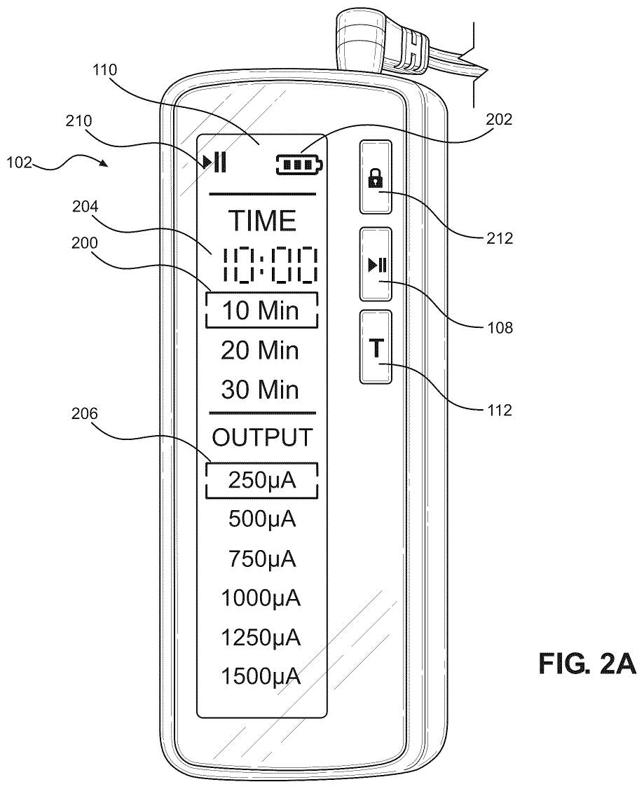

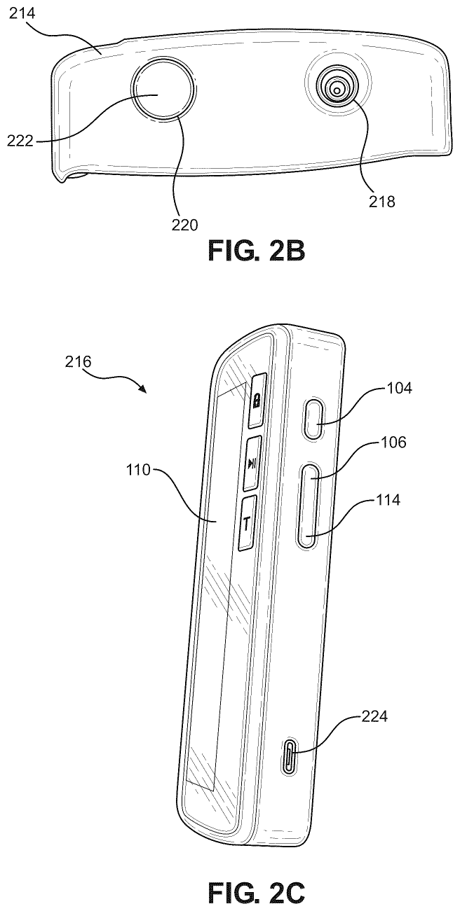

FIGS. 2A-2C illustrate a perspective view of another exemplary embodiment of the micro stimulation device 100. FIG. 2A illustrates a front panel 102, FIG. 2B illustrates a top panel 214, and FIG. 2C illustrates a side panel 216. The front panel 102 of the micro stimulation device 100 includes the display screen 110 which indicates a selection of treatment periods 200, a battery charge level indicator 202, a timer 204, and a selection of power levels 206. The display screen 110 may be lit when in use.

The micro stimulation device 100 also includes a start/pause button 108 and a start/pause indicator 210 on the display. In this embodiment, the micro stimulation device 100 further includes a lock button 212 that disables the other buttons. For example, when locked, the other buttons cannot be accidentally pressed to, e.g., increase a treatment period 200 or power level 206.

The top panel 214 includes at least one microcurrent output port 218 configured to output the generated microcurrent to one or more electrodes. A second microcurrent output port 220 may also be implemented. A top or cover 222 may fit over the output ports 218, 220 to prevent dust buildup in the ports or accidental current leakage.

The side panel 216 in this embodiment includes the power button 104. When activated, the power button 104 initiates the lighted display screen 110. The side panel 216 further includes an increase button 114 that is pressed/activated to increase the power level 206, and a decrease button 106 that is pressed/activated to decrease the power level 206. The micro stimulation device 100 may further include a rechargeable battery that is powered through a USB port 224, such as a USB-A, USB-B, USB-C, mini-USB, or micro-USB, though other types of power ports may be implemented.

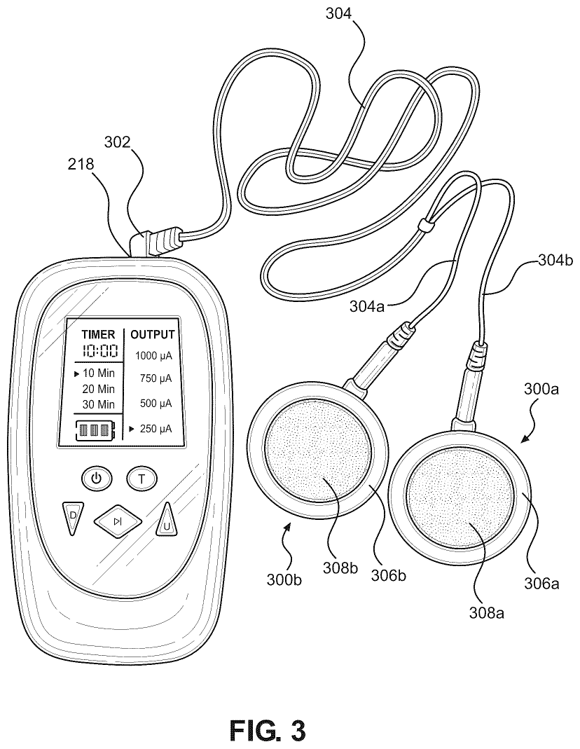

FIG. 3 illustrates a perspective view of an exemplary embodiment of the electrodes 300a-b of the micro stimulation device 100. An electrical connector 302 is configured for insertion into the microcurrent output port 218 of the micro stimulation device 100. The electrical connector 302 is electrically connected to lead wires 304, e.g., via the lead wires 304a, 304b, to at least two electrodes 300a, 300b. The microcurrent output is transmitted to each of the electrodes 300a, 300b by the two lead wires 304a, 304b. Each electrode 300a, 300b receives a microcurrent current output at the selected power level during the treatment period.

The electrodes 300a-b each include an electrode cup 306a, 306b and a replaceable electrode pad 308a, 308b. The electrode pads 308a-b include a spongelike material that directly contacts the targeted tissue. The electrodes pads 308a-b help to prevent possible skin burns or irritation from the microcurrent while providing gentle administration of microcurrent to the patient without undue discomfort. The electrode pads 308a-b may be single use and disposable. In another example, the electrode pads 308a-b may be designed for multiple uses, such as 10-14 uses or more.

For proper treatment, it is important that the electrodes 300a-b maintain conductivity to effectively deliver the desired electrical stimulation. One method for ensuring this conductivity is by applying a saline solution, e.g., a solution of sodium chloride in water, on the electrodes 300a-b. The saline solution helps to improve the contact between the electrodes 300a-b and the tissue and can also help to reduce the resistance between the electrodes 300a-b and the tissue, ensuring that the desired electrical signals are accurately and efficiently delivered to the skin of the patient. In use, the microcurrent from the micro stimulation device 100 spreads through the saline solution in the moistened electrode pads 308a-b and onto the skin of the patient in contact with the electrode pads 308a-b. By ensuring that the electrodes 300a-b have good electrical contact with the target tissue and that the electrical signals are accurately delivered, the saline solution helps to improve the overall efficacy of the stimulation and ultimately enhance the outcomes for patients.

One of the key advantages of micro stimulation for ocular treatment is its ability to directly target damaged or dysfunctional areas of the eye. Traditional treatments for conditions such as retinitis pigmentosa or macular degeneration often involve broad interventions such as laser therapy or injections, which can have limited efficacy and potential side effects. Micro stimulation, on the other hand, allows for precise and localized delivery of stimulation to the affected areas, potentially leading to more targeted and effective outcomes. By providing controlled stimulation to specific regions of the retina or other ocular structures, micro stimulation has shown great potential for improving vision in patients with conditions such as retinitis pigmentosa, age-related macular degeneration, and diabetic retinopathy. One of the key advantages of micro stimulation with saline electrode pads is its non-invasive nature, which makes it suitable for a wide range of patients, including those who may not be candidates for more invasive treatments. The use of saline as a conductive medium also helps to minimize skin irritation or discomfort, further improving the overall patient experience. Additionally, the targeted delivery of electrical signals can help to more effectively modulate neural pathways, leading to better outcomes for conditions such as chronic pain or neurological disorders. Additionally, because micro stimulation can be adjusted and personalized for each individual patient, it offers the potential for customized treatment plans that are tailored to specific needs, conditions, and/or responses of a patient.

FIGS. 4A-C illustrate perspective views of exemplary embodiments of various recommended placements of the electrodes 300 of the micro stimulation device 100 on a patient. Though the patient/user of the micro stimulation device 100 is shown as a human in these figures, the patient or user may also include other animals.

In FIG. 4A, a first electrode 300a (not shown) is positioned on the right temple of the patient, and a second electrode 300b is positioned on the left temple of the patient. The electrodes 300a-b contact the cutaneous tissue of the left and right temples and conduct the microcurrent from the micro stimulation device 100 to the skin tissue of the temples.

The electrodes 300a-b may be approximately 45-55 millimeters squared (mm²), e.g., each electrode sized as a square approximately 7 mm by 7 mm, or a circle having a diameter of approximately 7-9 mm. In another example, the electrodes 300a-b are approximately 36 mm², e.g., each electrode sized as a square approximately 6 mm by 6 mm, or a circle having a diameter of approximately 6 mm-7 mm. In another example, the electrodes may be approximately 25 mm² (e.g., each electrode sized as a square no larger than approximately 5 mm by 5 mm, or a circle having a diameter of approximately 5 mm-6 mm). The size of the electrodes 300a-b may be selected based on the type of condition being treated, the target position of the electrodes, the size of the patient's head, or other factors.

In FIG. 4B, the electrodes 300 are positioned over the right and left eyelid areas, e.g., covering the tissue of the eyelids while the eyes are closed. For example, the first electrode 300a is placed over the left eyelid and the second electrode 300b is placed over the right eyelid. The electrodes 300a-b contact the cutaneous tissue of the eyelids and conduct the microcurrent from the micro stimulation device 100 to the eyelid skin tissue.

In FIG. 4C, the electrodes 300 are positioned on the forehead of the patient. The first electrode 300a is centrally placed over the left eye above the left eyebrow, and the second electrode 300b is centrally placed over the right eye above the right eyebrow. The electrodes 300a-b contact the cutaneous tissue of the forehead and conduct the microcurrent from the micro stimulation device 100 to the forehead skin tissue.

In one embodiment, the first and second electrodes 300a, 300b may be held in position using an elastic headband. The clastic headband is positioned over the electrodes 300a-b and then is wrapped around the head. The clastic headband applies pressure to the electrodes 300a-b to hold them in position during treatment. In other embodiments, tape or an adhesive may be used to hold the electrodes 300a-b in position. The electrodes 300a-b are thus configured to contact and conduct the microcurrent from the micro stimulation device to the facial and/or ocular skin of a patient.

Though two electrodes 300a-b are illustrated in this embodiment, additional electrodes may be implemented. For example, two additional electrodes (not shown) may be connected to the micro stimulation device 100, e.g., using the second output port 220, and deliver current concurrently with the initial electrodes 300a-b. The additional electrodes may be positioned at a first and second targeted areas, as shown in FIGS. 4A-C, while the electrodes 300a-b are positioned at third and fourth different targeted areas shown in FIGS. 4A-C. The additional electrodes and the initial electrodes 300a-b may be of a same size or of different sizes. The use of additional electrodes 300 may increase the area of neural stimulation and may help in the treatment of the patient.

In addition, even though two electrodes 300a-b are illustrated in FIGS. 4A-C, a single electrode 300 may be used. For example, a patient may only have an ocular condition in a single eye. The patient may then prefer to position only a single electrode 300 in the targeted area to treat the eye with the ocular condition.

In addition, additional and/or alternate placement of the electrodes 300a-b may be directed for various treatments. For example, the target areas shown in FIGS. 4A-C may be adjusted or additional target areas for the electrodes 300a-b may be directed, such as under the eye sockets, around the cheekbones, in a nasal area, etc. The electrodes 300a-b may thus be placed on various target areas, including the cutaneous tissue of the face and eyelids.

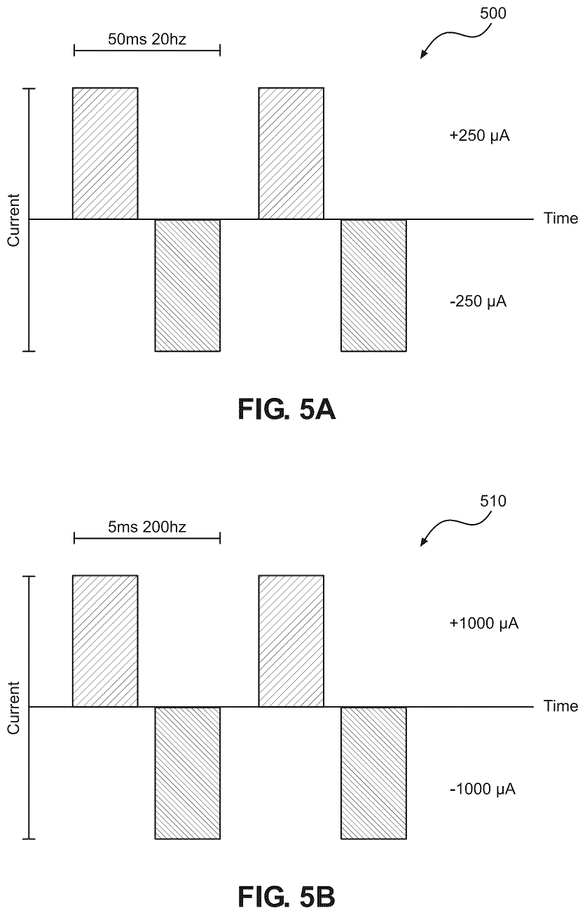

FIG. 5A illustrates a graph 500 of an embodiment of the microcurrent generated by the micro stimulation device 100 and delivered to each of the electrodes 300. The x-axis represents time and the y-axis represents current in micro amps (μA). In an embodiment, the micro stimulation device 100 generates an AC current with a frequency of approximately 20 hertz (Hz) which equates to one cycle of approximately 50 milliseconds (ms). The micro stimulation device 100 thus outputs 20 cycles per second with each cycle of approximately 50 ms. Tolerances of the micro stimulation device 100 are +/−10% for the frequency and pulse width. For example, the frequency may be in a range of 18 Hz to 22 Hz, and the pulse width may be in a range of 45 ms to 55 ms.

In this example, the AC power level is 250 μA but may also be set to 500 μA, 750 μA, 1000 μA, 1250 μA, 1500 μA or other value. In one example, these power levels are also within +/−10% tolerance. So, the AC power levels may include a first power level in a range of 225 μA to 275 μA, a second power level in a range of 450 μA to 550 μA, a third power level in a range of 675 μA to 725 μA, and a fourth power level in a range of 900 μA to 1100 μA, etc.

In another embodiment, the frequency of the AC current may be adjusted manually or automatically. For example, a user interface may be incorporated on the micro stimulation device 100 to increase or decrease the frequency of the AC current. For example, the AC current may be adjusted in frequency in first range between 20 Hz to about 200 Hz.

FIG. 5B illustrates a graph 510 of another embodiment of the microcurrent generated by the micro stimulation device 100 and delivered to each of the electrodes 300. The x-axis represents time and the y-axis represents current in micro amps (μA). In an embodiment, the micro stimulation device 100 generates an AC current with a frequency of approximately 200 hertz (Hz) which equates to one cycle of approximately 5 milliseconds (ms). The micro stimulation device 100 thus outputs 200 cycles per second with each cycle of approximately 5 ms. Tolerances of the micro stimulation device 100 are +/−10% for the frequency and pulse width. For example, the frequency may be in a range of 180 Hz to 220 Hz, and the pulse width may be in a range of 4.5 ms to 5.5 ms. In this example, the power level is set to 1000 μA with +/−10% tolerance. The micro stimulation device 100 may thus output a current with one of a plurality of selectable power levels and/or having one of a plurality of selectable frequencies.

The potential applications of micro stimulation devices for ocular treatment are broad and diverse. One promising area of application is in the treatment of retinal degenerative diseases, such as retinitis pigmentosa and age-related macular degeneration. By directly stimulating the remaining viable retinal cells, the micro stimulation device 100 may restore visual perception and improve quality of life for affected individuals. Additionally, the micro stimulation device 100 may treat optic nerve disorders, such as glaucoma and optic neuropathies, by modulating the neural activity at the level of the optic nerve head or along the visual pathway. The micro stimulation device 100 may complement other therapeutic approaches, such as pharmacological interventions and surgical procedures, and offer another avenues for personalized treatment strategies. For example, the conditions that may be treated by the micro stimulation device 100 include one or more of the following: Stargardt's syndrome, retinitis including retinitis pigmentosa, macular dystrophy, Best's disease, optic neuritis, Usher's syndrome, cone or rod dystrophy, Leber's syndrome, choroideremia, central retinal vein occlusion, branch retinal artery occlusion, central retinal artery occlusion, corneal lesions, cystoid macular edema, retinal vein occlusion, nystagmus, cataracts, amblyopia, histoplasmosis, toxoplasmosis, dry eye, surgical wounds, chorioretinopathy, corneal abrasions or wounds, retinopathy of prematurity, strabismus, refractive errors, facial nerve damage, and any ocular or facial nerve condition.

In alternative embodiments, micro stimulation device 100 may be used to maintain, optimize or improve vision for eyes that have no diagnosed pathology. For example, the micro stimulation device 100 may be used to preserve the health and integrity of the optic nerve by managing the host of underlying factors that contribute to possible degeneration of the optic nerve. A person at risk of an ocular condition, such as a diabetic patient, may use the micro stimulation device 100 to help maintain their eye health. Or in another example, a pilot with a need for optimal vision may use the micro stimulation device 100 to maintain or improve their ocular health.

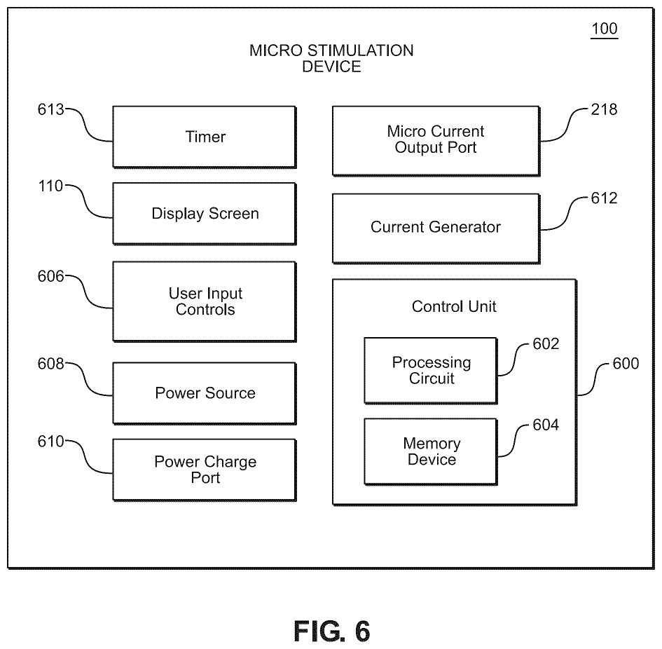

FIG. 6 illustrates a block diagram of an exemplary embodiment of the micro stimulation device 100. The micro stimulation device 100 includes a control unit 600 including a processing circuit 602 and memory device 604. The processing circuit 602 includes at least one processor, such as a central processor unit (CPU), microprocessor, microcontroller, embedded processor, digital signal processor, media processor, field programmable gate array, programmable logic device, state machine, logic circuitry, analog circuitry, digital circuitry, and/or any device that manipulates signals (analog and/or digital) based on hard coding of the circuitry and/or operational instructions.

The memory device 604 is a non-transitory memory and may be an internal memory or an external memory and may be a single memory or a plurality of memories. The memory device 604 may be a read-only memory, random access memory, volatile memory, non-volatile memory, static memory, dynamic memory, flash memory, cache memory, and/or any non-transitory memory device that stores digital information.

The memory device 604 stores computer-executable instructions which when executed by the processing circuit 602 causes the micro stimulation device 100 to perform one or more functions described herein. Computer-executable instructions may include, e.g., program modules such as routines, programs, objects, components, data structures, etc. that perform particular tasks or implement particular abstract data types. Such programs may be implemented in a high-level procedural or object-oriented programming language to communicate with a computer system. However, the program(s) can be implemented in assembly or machine language, if desired. In any case, the language may be a compiled or interpreted language, and combined with hardware implementations.

The micro stimulation device 100 includes a current generator 612 and power source 608. The power source 608 may include a battery, such as a 9 Volt battery, to power the micro stimulation device 100. The battery may be rechargeable and charged using a power charge port 610, e.g., when the power charge port 610 is connected to an electrical outlet or other power source, such as USB port 224. The micro stimulation device 100 may also be powered through the power charge port 610 when the battery lacks power or no battery is present. The power charge port 610 may also be configured to wirelessly charge the battery using a wireless charger.

The micro stimulation device 100 includes user input controls 606, such as the buttons described herein. Alternatively, or additionally, the user input controls 606 may include turnable knobs, touchscreen, keyboard, mouse, touchpad or other user input device. The micro stimulation device 100 also includes the display screen 110 that is configured to display the control settings.

The micro stimulation device 100 includes a timer circuit 613, such as a clock circuit, that tracks the duration of a treatment based on the treatment period 200 and is displayed as the timer 204. The micro stimulation device 100 generates the AC output for the duration of the treatment period 200 (unless paused and/or other stopped).

The micro stimulation device 100 includes a current generator 612 for generating a DC and/or AC output to the electrodes 300. The current generator 612 includes a DC to AC converter to convert the DC power from the battery to generate an AC microcurrent output. The current generator 612 is configured to generate the AC microcurrent output at one or more frequencies and/or having one or more power levels. The same AC microcurrent output is transmitted to each of the electrodes 300 through the microcurrent output port 218, such that each electrode 300 receives the same power level and frequency of microcurrent. The micro stimulation device 100 displays the AC output power level 206 and/or selected frequency on the display screen 110.

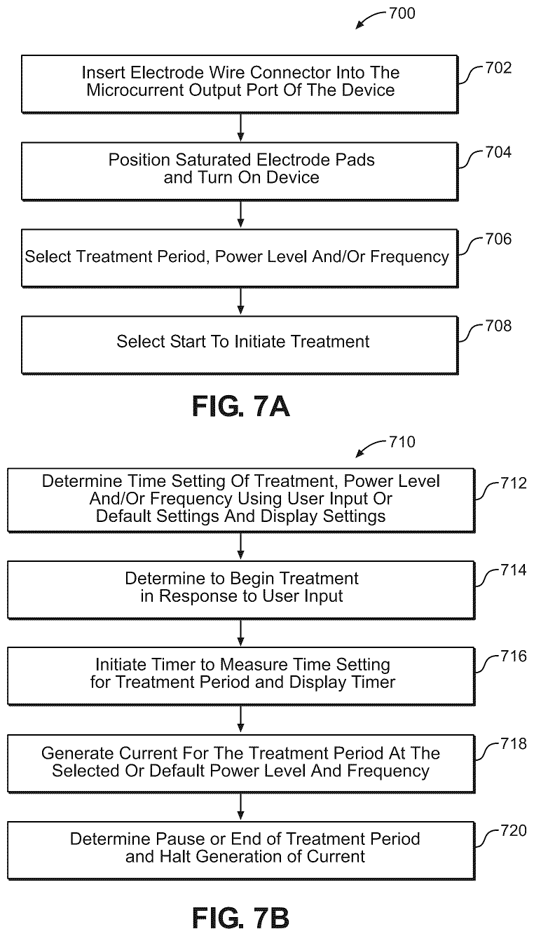

FIG. 7A illustrates a flow chart of an exemplary embodiment of a method 700 for initiation of operation of the micro stimulation device 100 by a user. In use, a first sponge is inserted into a first of two silicon electrodes 300, and a second sponge is inserted into a second of the two silicon electrodes 300. The electrodes 300 with the inserted sponges are immersed in or otherwise saturated with saline solution. Excess solution may be gently squeezed out.

The electrical connector 302 is then inserted into the microcurrent output port 218 of the micro stimulation device 100 at step 702 to connect the electrodes 300 to the micro stimulation device 100. The electrodes 300 are positioned at step 704 on the eyelids, forehead, or temple. The electrodes 300 may be held in position by a headband, tape, adhesive or other attachment means.

A user may select a treatment period 200, such as 10, 20 or 30 minutes, and a power level, such as 250 μA, 500 μA, 750 μA, 1000 μA, 1250 μA, 1500 μA at step 706. The user may also select one or more frequencies, such as 20 Hz, 50 Hz, 100 Hz, or 200 Hz at step 706. The user must then press the start/pausebutton 108 to initiate the current flow at step 708. A user may be instructed to perform the treatments once a day or twice a day.

FIG. 7B illustrates a flow chart of an exemplary embodiment of a method 710 of operation of the micro stimulation device 100. At step 712, the control unit 600 of the micro stimulation device 100 determines a treatment period, a power level and/or frequency selected by a user. When no treatment period, a power level and/or frequency has been selected by a user, then default settings are selected by the control unit 600. The control unit 600 controls the display to indicate the selected or default treatment period 200 and the selected or default power level 206 and/or frequency.

The control unit then waits to receive a start treatment selection from the user input controls 606 and determines to start treatment at step 714. The control unit 600 initiates the timer 204 to count down the selected treatment period 200 at step 716. The control unit 600 also signals the current generator 612 to generate the AC current at the selected or default power level and/or frequency at step 718. The current generator 612 generates a first microcurrent output that is transmitted to a first of the two electrodes 300, and a second microcurrent output that is transmitted to the second of the two electrodes 300. The first and second microcurrent outputs have the selected/default power level and frequency. The micro stimulation device 100 may automatically power off after a predetermined time (such as 2 minutes) when a completed circuit is not made with the electrodes 300a-b.

The current generator 612 continues to generate the AC current until the control unit 600 signals the current generator 612 to stop at step 720. The control unit 600 may signal the current generator 612 to stop in response to a user input of pause or when the timer 204 indicates the end of the treatment period.

In the foregoing specification, certain representative aspects have been described with reference to specific examples. Various modifications and changes may be made, however, without departing from the scope of the present invention as set forth in the claims. The specification and figures are illustrative, rather than restrictive, and modifications are intended to be included within the scope of the present invention. Accordingly, the scope of the invention should be determined by the claims and their legal equivalents rather than by merely the examples described. For example, the components and/or elements recited in any apparatus claims may be assembled or otherwise operationally configured in a variety of permutations and are accordingly not limited to the specific configuration recited in the claims. As such, the present teachings can be readily applied to other types of apparatuses and many alternatives, modifications, and variations will be apparent to those skilled in the art.

As may be used herein, the term “operable to” or “configurable to” indicates that an element includes one or more components, fasteners, or dimensions to perform one or more of the described or necessary corresponding functions and may further include inferred coupling to one or more other items to perform the described or necessary corresponding functions. As may also be used herein, the term(s) “coupled,” “coupled to,” “connected to” and/or “connecting” or “interconnecting” includes direct connection or and/or indirect connection through one or more other components. As may be used herein, the terms “substantially” and “approximately” provide an industry-accepted tolerance for its corresponding term and/or relativity between items.

As used herein, the terms “comprise,” “comprises,” “comprising,” “having,” “including,” “includes” or any variation thereof, are intended to reference a nonexclusive inclusion, such that a process, method, article, composition or apparatus that comprises a list of elements does not include only those elements recited but may also include other elements not expressly listed or inherent to such process, method, article, composition, or apparatus. Other combinations and/or modifications of the above-described structures, arrangements, applications, proportions, elements, materials, or components used in the practice of the present invention, in addition to those not specifically recited, may be varied, or otherwise particularly adapted to specific environments, manufacturing specifications, design parameters, or other operating requirements without departing from the general principles of the same.

Moreover, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is intended to be construed under the provisions of 35 U.S.C. § 112(f) as a “means-plus-function” type element, unless the element is expressly recited using the phrase “means for” or, in the case of a method claim, the element is recited using the phrase “step for.”

1. A micro stimulation device, comprising:
a current generator configured to generate a current; and
at least a first electrode electrically coupled to the micro stimulation device, wherein the at least first electrode is configured to contact and conduct the microcurrent to facial and/or ocular skin of a patient.

2. The micro stimulation device of claim 1, wherein the current generator is configured to generate the microcurrent at a selected one of a plurality of power levels, wherein the plurality of power levels includes: 250 micro amps (μA), 500 μA, 750 μA, 1000 μA, 1250 μA, and 1500 μA.

3. The micro stimulation device of claim 1, wherein the current generator is configured to generate the microcurrent at a selected one of a plurality of power levels, wherein the plurality of power levels includes: 250 micro amps (μA), 500 μA, 750 μA, and 1000 μA.

4. The micro stimulation device of claim 2, further comprising:
a first user input control configured for a user to select one of the plurality of power levels.

5. The micro stimulation device of claim 4, further comprising:
a control unit including a processing circuit and a memory device, wherein the memory device stores instructions that, when executed by the at least one processor, causes the micro stimulation device to:
determine the selected one of the plurality of power levels from the first user input control; and
control the current generator to generate the microcurrent at the selected one of the plurality of power levels.

6. The micro stimulation device of claim 4, further comprising:
a display; and
a graphical user interface generated on the display that indicates the selected one of the plurality of power levels.

7. The micro stimulation device of claim 6, further comprising:
a second user input control configured for a user to select one of a plurality of treatment periods, wherein the plurality of treatment periods includes: 10 minutes, 20 minutes, and 30 minutes.

8. The micro stimulation device of claim 7, wherein the control unit is further configured to cause the micro stimulation device to:
determine the selected one of the plurality of treatment periods from the second user input control; and
control the current generator to generate the microcurrent for a duration of the selected one of the plurality of time settings.

9. The micro stimulation device of claim 8, wherein the control unit is further configured to cause the micro stimulation device to:
generate a graphical user interface on the display that indicates the selected one of the plurality of treatment periods.

10. The micro stimulation device of claim 1, further comprising:
a rechargeable battery configured to power the micro stimulation device; and
a power charge port that is configured to connect to a power source and charge the rechargeable battery.

11. The micro stimulation device of claim 1, further comprising:
a microcurrent output port coupled to the current generator, wherein the microcurrent output port is coupled to the at least first electrode using a first lead wire.

12. The micro stimulation device of claim 11, further comprising:
at least a second electrode; and
a second lead wire coupled to the microcurrent output port and the second electrode.

13. The micro stimulation device of claim 12, wherein the first electrode and the second electrode each include:
an electrode cup; and
an electrode pad fitted within the electrode cup, wherein the electrode pad is removeable and replaceable from the electrode cup.

14. The micro stimulation device of claim 13, wherein the first electrode is positioned on and stimulates a right temple of a user with the microcurrent; and
wherein the second electrode is positioned on and stimulates a left temple of the user with the microcurrent.

15. The micro stimulation device of claim 13, wherein the first electrode is positioned on and stimulates a right eye area of a user with the microcurrent; and
wherein the second electrode is positioned on and stimulates a left eye area of the user with the microcurrent.

16. The micro stimulation device of claim 13, wherein the first electrode is positioned on and stimulates a right forehead area of a user with the microcurrent; and
wherein the second electrode is positioned on and stimulates a left forehead area of the user with the microcurrent.

17. The micro stimulation device of claim 1, further comprising:
a display with a graphical user interface that indicates a plurality of power levels, wherein the plurality of power levels includes: 250 micro amps (μA), 500 μA, 750 μA, 1000 μA, 1250 μA, and 1500 μA; and
a first user input control configured for a user to select one of the plurality of power levels on the display.

18. The micro stimulation device of claim 1, further comprising:
a display with a graphical user interface that indicates a plurality of treatment periods, wherein the plurality of treatment periods includes: 10 minutes, 20 minutes, and 30 minutes; and
a second user input control configured for a user to select one of the plurality of treatment periods on the display.

19. The micro stimulation device of claim 1, wherein the micro stimulation device is sized for portability and to be handheld.

20. The micro stimulation device of claim 19, wherein the micro stimulation device has a thickness of 12 mm to 16 mm, a height of 100 mm to 150 mm, and a width of 75 mm to 100 mm.
